(19) United States
(12) Patent Application Publication
(10) Pub. No.: US 2011/0313038 A1
(43) Pub. Date: Dec. 22, 2011

(54) CATIONIC OIL-IN-WATER EMULSIONS CONTAINING PROSTAGLANDINS AND USES THEREOF

(75) Inventors: Frederic Lallemand, Fresnes (FR); Betty Phillips, Antony (FR); Jean-Sebastien Garrigue, Verrieres le Buisson (FR)

(73) Assignee: NOVAGALI PHARMA SA, Evry (FR)

(21) Appl. No.: 13/203,754

(22) PCT Filed: Mar. 4, 2010
(86) PCT No.: PCT/EP2010/052741

§ 371 Date: Aug. 29, 2011

Related U.S. Application Data
(60) Provisional application No. 61/157,355, filed on Mar. 4, 2009.

(30) Foreign Application Priority Data
Mar. 4, 2009 (EP) 09305202.5

(51) Int. Cl. A61K 31/216; A61P 27/06; A61P 27/02; A61K 31/215; A61K 31/165

(52) U.S. Cl. 514/530; 514/622

(57) ABSTRACT

A colloidal cationic oil-in-water emulsion includes: —a prostaglandin, —an oil having a iodine value ≦2, —one or more surfactants including at least one quaternary ammonium compound, —water, wherein the prostaglandin/total sum of surfactants mass ratio is included between 0.5 and 5. The use of the cationic oil-in-water emulsions for enhancing the stability of the prostaglandins, for the treatment of ocular hypertension and/or glaucoma, for promoting growth of eyelashes and/or for treating eyelash hypotrichosis is also described.

The present invention pertains to cationic oil-in-water emulsions containing prostaglandins for the topical administration of prostaglandins and in particular for the treatment of ophthalmic conditions or diseases, preferably ophthalmic conditions affecting the interior of the eye, more specifically the anterior segment of the eye, including ocular hypertension and/or glaucoma, and also for promoting growth of eyelashes and/or for treating eyelash hypotrichosis. The cationic oil-in-water emulsion according to the invention further presents the advantage to enhance the chemical stability of prostaglandins.

In the present invention, the term “prostaglandin” is indifferently used for prostaglandins, their derivatives, precursors, prodrugs or analogues.

Glaucoma is a disease characterized by an increase in the intraocular pressure (IOP) and is often associated with optic nerve damage and visual field defect. If left untreated, glaucoma can ultimately lead to blindness.

Prostaglandins, especially prostaglandin F₂ₐₗₚₕₐ and its phenyl-substituted analogues, have been shown to effectively reduce the IOP in man and animals. In fact, they have been used in ophthalmic preparations in order to treat glaucoma. For instance, latanoprost is available in the form of a topical eye solution (eyedrops) and sold under the trademark Xalatan®.

Indeed, latanoprost is a potent prostaglandin F₂ₐₗₚₕₐ analogue which has been developed for the treatment of glaucoma. Its chemical name is isopropyl-(Z)-7-[(1R,2R,3R,5S)-3,5-dihydroxy-2-[(3R)₃-hydroxy-5-phenylpentyl]-cyclopentyl]-5-heptenoate, its molecular formula is C₂₆H₄₀O₅ and its chemical structure is:

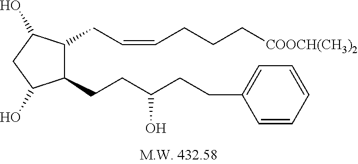

Specifically, latanoprost is a lipophilic prodrug in which the carboxylic acid moiety in the α-chain has been esterified to increase the bioavailability of the active drug into the eye. In addition, latanoprost is absorbed through the cornea where the isopropyl ester prodrug is hydrolyzed to the acid form to become biologically active.

Some ophthalmic prostaglandins, such as bimatoprost, latanoprost or travoprost, have also been described as being capable of promoting eyelash growth. Such prostaglandins could therefore be used for the topical treatment of eyelash hypotrichosis.

The problem generally encountered with prostaglandins is that they may be chemically unstable. In particular, latanoprost is known to be very sensitive towards light and heat. Indeed, these two elements (i.e. light and heat) may have an impact on the stability of latanoprost by provoking its hydrolyzation and/or oxidation. Consequently, unopened bottles of Xalatan® should be stored in the dark and under refrigeration at 2-8° C.

Consequently, there is a need for prostaglandin formulations which show an enhanced chemical stability of the prostaglandin and, in particular, an enhanced stability overtime towards light and heat.

The Applicant already conceived prostaglandin emulsions, and found that emulsions were a suitable vehicle to stabilize prostaglandins (see for example WO2007/042262).

However, there is a constant research for improvement in the field of ophthalmic emulsions, and the Applicant surprisingly experimented that stability of prostaglandins could be achieved with very low amounts of surfactants thus avoiding drawbacks due to high amounts of surfactants, often responsible of eye irritations or itching.

The present invention provides a prostaglandin composition which exhibits an improved stability of the prostaglandin compared to commercial products, while at the same time being light in surfactants, non toxic, tolerable for the patient and at least as efficient as the commercially available products.

An object of the present invention is a colloidal cationic oil-in-water emulsion comprising:
a prostaglandin,
an oil having a iodine value ≦2,
one or more surfactants including at least one quaternary ammonium compound,
water,
   wherein the prostaglandin/total sum of surfactants mass ratio is comprised between 0.5 and 5.

In particular embodiments of the present invention, the prostaglandin/total sum of surfactants mass ratio in the emulsion ranges from 0.5 to 4, or from 0.5 to 3, or ranges from 0.5 to 3, or ranges from 0.5 to 2, or ranges from 0.6 to 1.5, or ranges from 0.7 to 1.3, or is around 1. In the meaning of this invention, the term “around” preceding a selected value means ranging from plus or minus 10% of the selected value.

According to an embodiment, the emulsion includes prostaglandin, oil and surfactant(s), and is such that the ratio of the oil mass to the total mass of surfactants ranges from 50 to 500, preferably 100 to 400, more preferably around 200.

According to a preferred embodiment, in the emulsion, the prostaglandin/total sum of surfactants mass ratio ranges from 0.5 to 4, and the oil/total sum of surfactants mass ratio ranges from 50 to 500.

Surprisingly, the emulsions of the present invention show a remarkable stability although they contain very low amounts of surfactants and consequently an enhanced tolerability for patients.

According to the invention, “good tolerability” or “enhanced tolerability” means that the ratio “therapeutic benefit” to “ocular discomfort” is acceptable by the patient, and preferably similar to a placebo or NaCl solution 0.9%. It is generally accepted that in order to show good ocular tolerability the cationic agent content within the formulation should not exceed 0.1%, preferably not exceed 0.05% and even more preferably should not exceed 0.03%.

According to a preferred embodiment, the emulsion according to the present invention comprises one or more surfactants, including at least one quaternary ammonium compound.

In the emulsion according to the present invention, the quaternary ammonium compound is used as a cationic agent. It is preferably chosen among cetalkonium halide, benzalkonium halide, lauralkonium halide, cetrimide, hexadecyltrimethylammonium halide, tetradecyltrimethylammonium halide, dodecyltrimethylammonium halide, cetrimonium halide, benzethonium halide, behenalkonium halide, cetethyldimonium halide, cetylpyridinium halide, benzododecinium halide, chlorallyl methenamine halide, myristylalkonium halide, stearalkonium halide or mixtures thereof. Halide includes bromide and chloride.

Preferably, the quaternary ammonium compound is chosen among cetalkonium chloride, benzalkonium chloride or mixtures thereof.

In another embodiment of the invention, the emulsion comprises one or more quaternary ammonium compounds as the only surfactant present in the emulsion. In other words, in this embodiment, the total sum of the surfactants in the emulsion consists of the sum of all quaternary ammonium compound(s).

According to the invention, the emulsion includes only one surfactant, which is a quaternary ammonium compound, preferably cetalkonium chloride. In this embodiment, the emulsion of the invention is a more simple emulsion than in prior art, i.e. containing less ingredients than in prior art emulsions.

The concentration of the quaternary ammonium compound, preferably of cetalkonium chloride, is preferably comprised between 0.001 and 0.1% w/w, more preferably between 0.002 and 0.05% w/w and even more preferably between 0.002 and 0.03% w/w.

Typically, in addition to quaternary ammonium surfactants, the emulsion may include further surfactants, such as for example further cationic surfactants, anionic surfactants, nonionic surfactants, zwitterionic surfactants, hydrophilic surfactants (with a high HLB), hydrophobic surfactants (with a low HLB) or mixtures thereof, as long as the zeta potential of the invention remains positive.

The further cationic surfactants comprise C10-C24 primary alkylamines, such as oleylamine or stearylamine, tertiary aliphatic amines, cationic lipids, amino alcohols, biguanide salts chosen from chlorhexidine and salts thereof, polyaminopropyl biguanide, phenformin, alkylbiguanide or mixtures thereof, cationic polymers selected from chitosan, 1,2-dioleyl-3-trimethylammonium-propane, 1, 2-dioleoyl-sn-glycero-phosphatidylethanolamine, cationic glycosphingo-lipids or cationic cholesterol derivatives, or mixtures thereof.

Typically, the nonionic surfactants comprise alkyl polyethylene oxide, alkylphenol polyethylene oxide, poloxamers, tyloxapol, alkyl polyglucosides, fatty alcohols, cocamide MEA, cocamide DEA, polyoxyethylene castor oil derivatives, sorbitan esters, polyoxyl stearates, polysorbates or mixtures thereof. According to another embodiment, the composition of the invention is free of polyoxyethylene castor oil derivatives.

Typically, the anionic surfactants comprise perfluorooctanoate, perfluorooctanesulfonate, alkyl sulphate salts, sodium lauryl ether sulphate, alkyl benzene sulfonate, soaps or fatty acid salts or mixtures thereof.

Typically, the zwitterionic surfactants comprise dodecyl betaine, cocamidopropyl betaine, coco-ampho-glycinate or mixtures thereof.

Typically, the surfactant according to the invention comprises hydrophilic surfactants (with a high HLB) and/or hydrophobic surfactant (with a low HLB) or mixtures thereof.

Preferably, the emulsion is free of phospholipids.

In a particular embodiment, the surfactants present in the emulsion of the invention are chosen among quaternary ammonium compounds, poloxamers, tyloxapol, polysorbates, sorbitan esters, polyoxyl stearates or mixtures thereof.

In a particular embodiment, the emulsion comprises latanoprost, medium chain triglycerides (MCT), cetalkonium chloride as the only surfactant.

The emulsion according to the invention is a cationic emulsion, which means that the zeta potential remains positive overtime. This positive zeta potential may preferably be higher than 10 mV, more preferably be higher or equal to 20 mV.

It has long been recognised that the zeta potential is a very good index of the magnitude of the interaction between colloidal particles and measurements of zeta potential are commonly used to assess the stability of colloidal systems. The zeta potential measured in a particular system is dependent on the chemistry of the surface, and also of the way it interacts with its surrounding environment.

Typically, the cationic emulsions according to the invention are physically stable overtime and may keep a positive zeta potential over a period of two years at 25° C. For each measurement of the zeta potential, it is operated as follows:

The zeta potential of the emulsion droplet surface is determined by electrophoretic mobility in an apparatus such as a Malvern Zetasizer 2000 (Malvern Instruments, UK) equipped with suitable software and calibrated with the supplied standard.

The emulsion is diluted in double distilled water if needed in order to obtain the scattering intensity allowing optimal particle detection. The sample count rate should be between 100 to 1000 KCps, in homodyne detection (if heterodyne detection is used, the contribution of the reference beam should be deduced). Three consecutive measurements are performed at 25° C. using a constant cell drive of 150 mV. The electrophoretic mobility is converted into zeta potential values through the Smoluchowsky equation, using the dielectric constants and viscosity of water. The measured value corresponds to the average of the 3 obtained values.

In a particular embodiment, the emulsion of the invention is free of any buffer.

According to a particular embodiment of the present invention, the emulsion remains stable during autoclaving. According to the present invention, “autoclaving” is defined as sterilization of a product by steam under pressure, by heating said product in an autoclave at high temperatures (e.g. 100 to 200° C., preferably 121° C.) during an extended period of time (e.g. 10 to 60 minutes, preferably 10 to 20 minutes) at around 103 kPa (15 psi) above atmospheric pressure. The steam and pressure transfer sufficient heat into organisms to kill them and thus sterilize the product.

According to the invention, “stability” is defined as the extent to which a product retains, within specified limits and throughout its period of storage and use (i.e., its shelf life), the same properties and characteristics that it possessed at the time of manufacture.

The purpose of stability testing is to provide evidence concerning the quality of a drug substance or a drug product overtime, said product being subjected to a variety of environmental factors such as temperature, humidity and light. The result may be helpful in providing appropriate storage conditions, re-testing periods and shelf lives.

Although conventional stability studies do evaluate those factors which ultimately affect the expiration date of the drugs, these conventional studies are time and cost-consuming. Consequently, in order to predict shelf-life of a pharmaceutical product for example, the pharmaceutical industry usually uses “accelerated stability studies” (Stress Test). These accelerated studies help understand the intrinsic stability mechanism of the molecule of interest by establishing degradation pathways and by identifying the likely degradation products. In these types of studies, the products are usually subjected to extreme conditions, such as temperature of about 40° C. for approximately 6 months.

According to the invention, “colloidal” means that the emulsion comprises colloid particles having an oily core surrounded by an interfacial film dispersed in water with a particle size equal or less than 1 μm. Typically, the oily core comprises a prostaglandin and an oil. The prostaglandin being lipophilic, it is thus understandable that it is essentially present in the oily core. Typically, the emulsion may contain other ingredients, such as emollients, preferably glycerol, pH adjusters, such as NaOH, osmotic agents or preservatives.

The cationic emulsion according to the invention such that the colloidal particles have an average particle size of equal or less than 1 μm, advantageously equal or less than 300 nm, more advantageously in the range of 100 to 250 nm.

In one embodiment, the prostaglandin is a prostaglandin F₂ₐₗₚₕₐ, a derivative, precursor, prodrug or analogue thereof.

In another embodiment, the prostaglandin is an ophthalmic prostaglandin, in particular a prostaglandin which is effective in treating ocular hypertension and/or glaucoma.

In another embodiment, the prostaglandin is an ester prodrug, an amide prodrug of an active prostaglandin, in particular of an active ophthalmic prostaglandin or a mixture thereof. Ester prodrugs include C₁-C₄ alkyl ester prodrugs, such as methyl ester, ethyl ester, isopropyl ester or butyl ester and amide prodrugs include C₁-C₄ alkyl amide prodrugs, such as methyl amide, ethyl amide, isopropyl amide or butyl amide.

According to a particular embodiment, the prostaglandin of the present invention is chosen among latanoprost, isopropyl unoprostone, travoprost, bimatoprost, tafluprost, or mixtures thereof.

In a preferred embodiment, the emulsion according to the present invention comprises latanoprost.

The amount of prostaglandin present in the oily core of the emulsion according to the invention depends on the nature of the prostaglandin and to the intended use. Typically, the amount of prostaglandin relative to the total weight of the emulsion is comprised between 0.001 to 1% w/w, preferably ranging from 0.002 to 0.3% w/w and even more preferably from 0.004 to 0.15% w/w.

In a particular embodiment, the prostaglandin can be combined with other antiglaucoma active ingredients, such as dorzolamide or timolol.

According to the present invention, the oil is preferably chosen among saturated oils which are able to limit the degradation of the prostaglandin by oxidation and/or hydrolysis in the emulsion.

According to the invention “saturated oil” is an oil which has an iodine value of less or equal to 2, preferably less than 2, which means that the oil is substantially free of any molecule having a hydrocarbon chain containing double or triple bonds.

The iodine value can be measured for example according to the methods disclosed in the European Pharmacopeia monograph 2.5.4 or US Pharmacopeia monograph 401.

According to a particular embodiment of the present invention, the oil is chosen among oily fatty acids, oily fatty alcohols, fatty acids esters, such as isopropyl myristate and isopropyl palmitate, vegetable oils, animal oils, mineral oils such as petrolatum, liquid paraffin, semi-synthetic oils such as fractionated oils obtained from vegetable oils or mixtures thereof.

According to the invention “semi-synthetic oils” are prepared by chemical synthesis from natural oils.

Particularly, the oil according to the invention is a semi-synthetic oil obtained from fractionated coconut oil, kernel oil or babassu oil. More particularly, the oil is medium chain triglycerides (MCT).

Indeed, according to the European Pharmacopeia, medium chain triglycerides (MCT) is described as the fixed oil extracted from the hard, dried fraction of the endosperm of Cocos nucifera L. by hydrolysis, fractionation of the fatty acids obtained, and re-esterification. MCT consists of a mixture of exclusively short- or medium-chain triglycerides of fatty acids, of which not less than 95% are the saturated fatty acids octanoic (caprylic) acid and decanoic (capric) acid.

Moreover, MCT can also be found in substantial amounts in kernel oil and babassu oil, in addition to some animal products, such as milk-fat, which may contain small amounts (up to 4%) of MCT.

In some embodiments, the amount of the oil relative to the total weight of the emulsion is not higher than 7% w/w, preferably between 0.5 and 5% w/w and even more preferably between 1 and 3% w/w.

In another embodiment, the pH of the emulsion is preferably comprised between 4 and 7, preferably ranging from 4.5 and 6.5 and more preferably ranging from 4.5 and 6, even more preferably around 5.

According to a preferred embodiment, the composition of the invention includes an osmotic agent, preferably glycerol.

Another object of the present invention is a process for manufacturing the emulsion previously described. The emulsions of the invention are therefore prepared according to the following steps:
preparation of the oily phase by mixing the prostaglandin (e.g. latanoprost) with the saturated oil (e.g. MCT) and possibly the quaternary ammonium when it is not soluble in the aqueous phase,
preparation of the aqueous phase by mixing the water-soluble ingredients (e.g. glycerol,) with purified water;
incorporating the oily phase to the aqueous phase;
decreasing the emulsion droplet size by any suitable means known to the man skilled in the art, for example by high-shear mixing;
homogenizing the cooled emulsion;
optionally, adjusting the pH to a physiological pH, by using for example NaOH or HCl;
optionally sterilizing the emulsion by autoclaving.

An additional advantage of the emulsions of the invention is that they are stable during the autoclaving, in other words, the prostaglandin is not degraded and the zeta potential remains positive.

According to the present invention, the cationic oil-in-water emulsion according to the invention is useful for the treatment of patients suffering from an ophthalmic condition or disease, preferably an ophthalmic condition or disease of the interior of the eye. In an embodiment of the invention, said patients are not suffering from lesions of the ocular surface, especially of the cornea or the conjunctiva. In the meaning of this invention, a corneal or conjunctival lesion is a local destruction of corneal, conjunctival or goblet cells. Such lesions may be local or disseminated and result in corneal erosion, punctuate keratopathy, epithelial defects, corneal ulceration, corneal scarring, corneal thinning, corneal perforation, keratitis, conjunctivitis, wounds, tiny abrasions, etc.

In another embodiment, the patient is not intolerant to quaternary ammonium halides.

The emulsion according to the present invention is preferably intended to be applied topically, e.g. to the surface of the eye, especially on cornea or conjunctiva, or to hairs, such as eyelashes of a patient.

Thus, the present invention is also directed to a method for treating a patient suffering from an ophthalmic condition or disease, preferably an ophthalmic condition or disease of the interior of the eye, by administering a therapeutic amount of the composition of the invention. The dose regimen being two drops every day or one drop every day on the eye surface. This treatment is a lifelong treatment.

According to the present invention, the cationic oil-in-water emulsion is useful for promoting growth of eyelashes or treating eyelash hypotrichosis.

According to an embodiment of the invention, the emulsion may be in the form of eye drops, eye ointment, or ophthalmic gel.

An object of the present invention is a delivery device comprising the cationic oil-in-water emulsion according to the invention.

Typically the delivery device according to the invention is selected from the group comprising lenses, ocular patch, implant, insert.

Other features and advantages of the invention will emerge upon reading the following non limiting examples.

### BRIEF DESCRIPTION OF THE FIGURES

FIG. 1: Latanoprost free acid concentration in ciliary body and in cornea after administration of the emulsion of the invention.

### EXAMPLES

### 1. Preparation of a Cationic Oil-in-Water Emulsion

The cationic oil-in-water emulsion according to the present invention is prepared by the following steps:
preparation of the oily phase by mixing at 50° C. the prostaglandin (latanoprost) with the saturated oil (MCT) and cetalkonium chloride respecting mass the ratio prostaglandin/total sum of surfactants of 1 and the ratio oil mass to the total mass of surfactants of 200.
preparation of the aqueous phase by mixing at 50° C. glycerol and purified water;
incorporating the oily phase to the aqueous phase;
decreasing the emulsion droplet size by any suitable means known to the man skilled in the art, for example by high-shear mixing 5 min at 16 000 rpm (Polytron PT6100; Kinematica, Switzerland)
homogenizing during 20 min at 15 000 psi the cooled emulsion (Emulsiflex C3, Avestin, Canada)
adjusting the pH with NaOH
sterilizing the emulsion by autoclaving.

A composition of the emulsion is given in table 1.

TABLE 1

| | | | Theorethical |
|---|---|---|---|
| | | | composition |
| | Ingredients | Supplier | (% w/w) |
| Oily phase | MCT (Medium Chain | Sasol GmbH, | 1.000 |
| | Triglycerides) | Germany | |
| | Latanoprost | | 0.005 |
| | cetalkonium chloride | Dishman, India | 0.005 |
| Aqueous | Glycerol | Merck, Germany | 2.400 |
| phase | Water (up to 100) | | 96.590 |
| | NaOH 1M | Merck, Germany | qs pH 7 |
| | Total | | 100% |

After sterilisation, the pH of the emulsion is around 5.0.

TABLE 2

| | | | Theorethical |
|---|---|---|---|
| | | | composition |
| | Ingredients | Supplier | (% w/w) |
| Oily phase | MCT (Medium Chain | Sasol GmbH, | 1.000 |
| | Triglycerides) | Germany | |
| | Travoprost | | 0.004 |
| | Cetalkonium chloride | Dishman, India | 0.005 |
| Aqueous | Glycerol | Merck, Germany | 2.400 |
| phase | Water (up to 100) | | 96.590 |
| | NaOH 1M | Merck, Germany | qs pH 7 |
| | Total | | 100% |

### 2. Stability Test & Comparative Test

The stability of the emulsion of example 1 was evaluated under accelerated conditions “Stress Test” (at 80° C. during 14 days), while a comparative analysis was conducted between the cationic emulsion (invention) and Xalatan® under the same “Stress Test” conditions. Prostaglandin content was analysed in both tests by an HPLC-UV method. The results are given in table 2 (stability test) and table 3 (comparative test).

TABLE 3

| Emulsion of | | Zeta potential | Osmolality | | Droplet | Latanoprost |
|---|---|---|---|---|---|---|
| example 1 | Aspect | (mV) | (mOsm/kg) | pH | size (nm) | (% w/w) |
| T = 0 days | White milky | 55.2 | 280 | 4.9 | 188 | 0.00511 |
| | homogeneous | | | | | |
| | emulsion | | | | | |
| | Tyndall effect | | | | | |
| T = 7 days | White milky | 44.5 | 277 | 4.86 | 208 | — |
| | homogeneous | | | | | |
| | emulsion | | | | | |
| | Tyndall effect | | | | | |
| T = 14 days | White milky | 42.5 | 281 | 4.88 | 221 | 0.00510 |
| | homogeneous | | | | | |
| | emulsion | | | | | |
| | Tyndall effect | | | | | |

TABLE 4

| | Latanoprost | | | | Zeta potential | |
|---|---|---|---|---|---|---|
| | (% w/w) | | pH | | (mV) | |
| | T0 | T14 days | T0 | T14 days | T0 | T14 days |
| Emulsion of | 0.00511 | 0.00492 | 4.9 | 4.88 | 55.2 | 42.5 |
| example 1 | | | | | | |
| Xalatan ® | 0.00510 | 0.00248 | 6.74 | 6.71 | NA | NA |

NA: not applicable

At T0, the concentrations in prostaglandins for the emulsion (invention) and for Xalatan® are close to 0.005%. However, after subjecting both emulsions to the “Stress Test” (14 days at 80° C.), it can be observed that the concentration of prostaglandins remains the same for the emulsion (invention), while it has decreased by more than half in the case of Xalatan®. Furthermore, surprisingly, with a ratio of the oil mass to the total mass of surfactants of 200 (there is 200 times more oil than surfactant), the emulsion did not cream, did not coalesce nor separate.

### 3. Pharmacokinetic/Pharmacodynamic Studies of the Emulsion of Table 1

Male and female New Zealand White rabbits were administrated with the emulsion of Table 1 or Xalatan® and latanoprost free acid concentration was determined at different time points after administration (0.25, 0.5, 1, 4, 6 and 24 hour(s)) at the following target tissues: conjunctiva, cornea, aqueous humor and ciliary body. Tₘₐₓ and AUC 0.5-24 h were calculated and are presented hereafter in Table 5.

TABLE 5

| | Numerical | | Reference | Cationic |
|---|---|---|---|---|
| | field name | Units | XALATAN ® | emulsion |
| Aqueous | Tₘₐₓ | h | 0.5-1 | 0.5-1 |
| humor | AUC_{0.25-24 h} | pg × h/μl | 327 | 344 |
| Cornea | Tₘₐₓ | h | 0-0.25 | 0.5-1 |
| | AUC_{0.25-24 h} | pg × h/mg | 2623 | 1925 |
| Conjunctiva | Tₘₐₓ | h | 0.25-0.5 | 0.5-1 |
| | AUC_{0.25-24 h} | pg × h/mg | 197 | 174 |
| Ciliary body | Tₘₐₓ | h | 0-0.25 | 0.25-0.5 |
| | AUC_{0.25-24 h} | pg × h/mg | 295 | 314 |

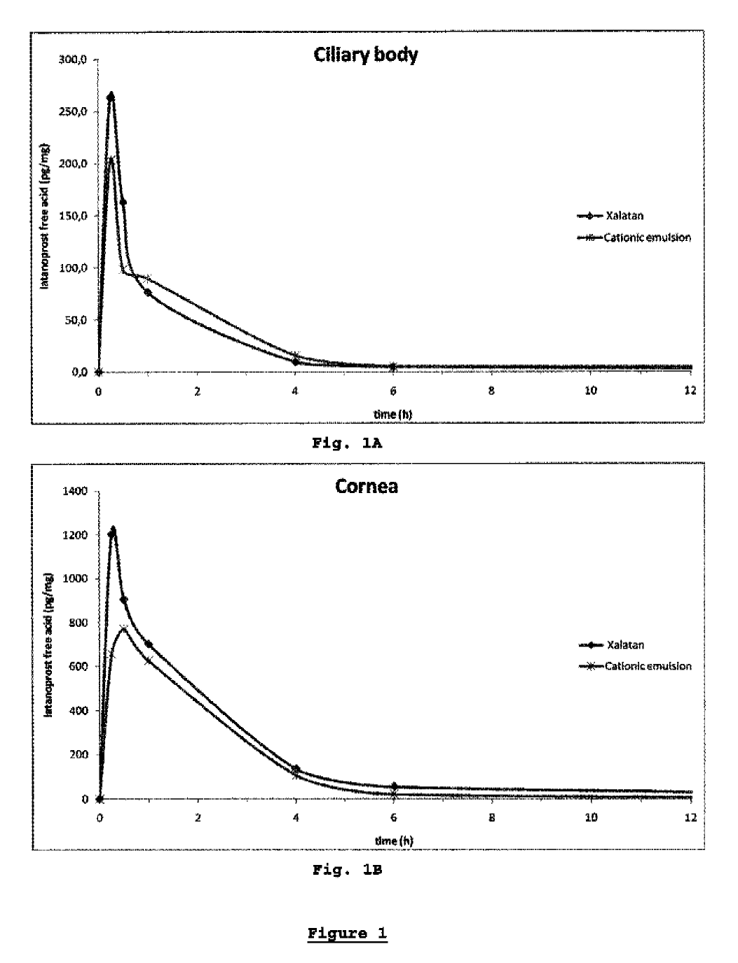

FIG. 1 (ciliary body and cornea) and results here above presented show that latanoprost free acid is present at a high concentration in the target ocular tissues after administration of the emulsion. Said concentrations are similar to Xalatan(r) concentrations and are known to be sufficient to allow the opening of the Schlemm's canal and thus the evacuation of aqueous humor, thereby reducing the intraocular pressure.

1-15. (canceled)

16. An oil-in-water emulsion comprising:
a prostaglandin,
an oil having a iodine value ≦2,
one or more surfactants including at least one quaternary ammonium compound,
water,
wherein the emulsion has a positive zeta potential, and the prostaglandin/total sum of surfactants mass ratio is comprised between 0.5 and 5.

17. The oil-in-water emulsion according to claim 16, wherein the prostaglandin/total sum of surfactants mass ratio is comprised between 0.5 and 4, preferably between 0.5 and 3, more preferably between 0.5 and 2, even more preferably between 0.6 and 1.5, even more preferably between 0.7 and 1.3, even more preferably around 1.

18. The oil-in-water emulsion according to claim 16, wherein the prostaglandin is chosen among an ester prodrug, an amide prodrug of an active prostaglandin or mixtures thereof.

19. The oil-in-water emulsion according to claim 16, wherein the prostaglandin is chosen among latanoprost, isopropyl unoprostone, travoprost, bimatoprost, tafluprost, or mixtures thereof.

20. The oil-in-water emulsion according to claim 16, wherein the quaternary ammonium compound is chosen among cetalkonium halide, benzalkonium halide, lauralkonium halide, cetrimide, hexadecyltrimethylammonium halide, tetradecyltrimethylammonium halide, dodecyltrimethylammonium halide, cetrimonium halide, benzethonium halide, behenalkonium halide, cetethyldimonium halide, cetylpyridinium halide, benzododecinium halide, chlorallyl methenamine halide, myristylalkonium halide, stearalkonium halide or mixtures thereof.

21. The oil-in-water emulsion according to claim 16, wherein the quaternary ammonium compound is chosen among cetalkonium chloride, benzalkonium chloride or mixtures thereof.

22. The oil-in-water emulsion according to claim 16, wherein the oil is chosen among oily fatty acids, oily fatty alcohols, fatty acids esters, such as isopropyl myristate and isopropyl palmitate, vegetable oils, animal oils, mineral oils such as petrolatum, liquid paraffin, semi-synthetic oils such as fractionated oils obtained from vegetable oils or mixtures thereof.

23. The oil-in-water emulsion according to claim 22, wherein the fractionated oil is medium chain triglycerides (MCT).

24. The oil-in-water emulsion according to claim 16, wherein the surfactants are chosen among quaternary ammonium compounds, poloxamers, tyloxapol, polysorbates, sorbitan esters, polyoxyl stearates or mixtures thereof.

25. The oil-in-water emulsion according to claim 16, wherein the amount of prostaglandin relative to the total weight of the emulsion is comprised between 0.001 to 1% w/w, preferably between 0.002 to 0.3% w/w and even more preferably between 0.004 to 0.15% w/w.

26. The oil-in-water emulsion according to claim 16, wherein the amount of the quaternary ammonium compound relative to the total composition is comprised between 0.001 and 0.1% w/w, preferably between 0.002 and 0.05% w/w and even more preferably between 0.002 and 0.03% w/w.

27. The oil-in-water emulsion according to claim 16, wherein the amount of the oil relative to the total weight of the emulsion is not higher than 7% w/w, preferably between 0.5 and 5% w/w and even more preferably between 1 and 3% w/w.

28. The oil-in-water emulsion according to claim 16, wherein the prostaglandin/total sum of surfactants mass ratio ranges from 0.5 to 4, and the oil/total sum of surfactants mass ratio ranges from 50 to 500.

29. A method for treating ocular hypertension and/or glaucoma comprising administering to a subject in need thereof an oil-in-water emulsion comprising:
a prostaglandin,
an oil having a iodine value ≦2,
one or more surfactants including at least one quaternary ammonium compound,
water,
wherein the emulsion has a positive zeta potential, and the prostaglandin/total sum of surfactants mass ratio is comprised between 0.5 and 5.

30. The method according to claim 29, wherein the prostaglandin/total sum of surfactants mass ratio is comprised between 0.5 and 4, preferably between 0.5 and 3, more preferably between 0.5 and 2, even more preferably between 0.6 and 1.5, even more preferably between 0.7 and 1.3, even more preferably around 1.

31. The method according to claim 29, wherein the prostaglandin is chosen among latanoprost, isopropyl unoprostone, travoprost, bimatoprost, tafluprost, or mixtures thereof.

32. The method according to claim 29, wherein the quaternary ammonium compound is chosen among cetalkonium halide, benzalkonium halide, lauralkonium halide, cetrimide, hexadecyltrimethylammonium halide, tetradecyltrimethylammonium halide, dodecyltrimethylammonium halide, cetrimonium halide, benzethonium halide, behenalkonium halide, cetethyldimonium halide, cetylpyridinium halide, benzododecinium halide, chlorallyl methenamine halide, myristylalkonium halide, stearalkonium halide or mixtures thereof.

33. A delivery device comprising an oil-in-water emulsion comprising:
a prostaglandin,
an oil having a iodine value ≦2,
one or more surfactants including at least one quaternary ammonium compound,
water,
wherein the emulsion has a positive zeta potential, and the prostaglandin/total sum of surfactants mass ratio is comprised between 0.5 and 5.
